(19) United States
(12) Patent Application Publication
(10) Pub. No.: US 2023/0372130 A1
(43) Pub. Date: Nov. 23, 2023

(54) STENT

(71) Applicant: Japan Lifeline Co., Ltd., Tokyo (JP)

(72) Inventors: Eiichi NAKANO, Tokyo (JP); Masamune SAKAI, Tokyo (JP); Fumiaki KOBAYASHI, Tokyo (JP)

(21) Appl. No.: 18/363,238

(22) Filed: Aug. 1, 2023

Related U.S. Application Data
(63) Continuation of application No. PCT/JP2021/011857, filed on Mar. 23, 2021.

(51) Int. Cl. A61F 2/90

(52) U.S. Cl. CPC A61F 2/90

(57) ABSTRACT

An object is to provide a stent having, in a well-balanced manner, a high expansion force that enables the stent to come into close contact with an inner wall of a tubular organ, such as a digestive tract, to sufficiently expand a constricted part and good flexibility by which none of an ulcer and a perforation is generated at sites with which both end parts of the stent come into contact even when the stent is placed in a bent tubular organ. A stent of the disclosure is a stent formed in a tubular shape by braiding one or more wire rods. A radial force (RF) ranges from 0.02 N/mm to 0.04 N/mm, and a ratio (RF/AF) of the radial force (RF) to an axial force (AF) is 0.14 mm⁻¹ or more. The stent of the disclosure preferably has a shortening of 35% or less.

### CROSS REFERENCE TO RELATED APPLICATIONS

This application is a continuation application of International Application No. PCT/JP2021/011857, filed on Mar. 23, 2021, the entire contents of which are hereby incorporated by reference.

### TECHNICAL FIELD

The disclosure relates to a stent to be placed in a tubular organ in a body, such as a digestive tract, to prevent stenosis and occlusion of the tubular organ.

### BACKGROUND

A stent to be placed in the digestive tract (digestive tract stent) is used to expand a lumen of the digestive tract constricted by a tumor.

The digestive tract stent is formed in a tubular shape by braiding one or more wire rods and has a mesh structure formed by connecting, along an axial direction, a plurality of circumferential units in which a plurality of meshes is arrayed along the circumferential direction (see, for example, JP 2009-501049 T below).

### SUMMARY

The digestive tract stent is required to have a high expansion force that enables the digestive tract stent to come into close contact with an inner wall of a digestive tract to sufficiently expand a constricted part.

Meanwhile, the digestive tract stent is also required to have flexibility such that both end parts of the digestive tract stent do not press the inner wall of the digestive tract to cause an ulcer or a perforation when placed in a bent digestive tract.

The digestive tract stent is further required to have a small so-called shortening (rate of decrease in length accompanying expansion).

However, known digestive tract stents do not satisfy all the above requirements, for example, a stent having a high expansion force tends to have inferior flexibility, and a stent having a good flexibility tends not to have a sufficient expansion force.

The disclosure has been made on the basis of the above circumstances.

An object of the disclosure is to provide a stent having, in a well-balanced manner, a high expansion force that enables the stent to come into close contact with an inner wall of a tubular organ to sufficiently expand a constricted part and good flexibility by which none of an ulcer and a perforation are generated at sites with which both end parts of the stent come into contact even when the stent is placed in a bent tubular organ.

Another object of the disclosure is to further provide a stent having a small shortening.
(1) A stent of the disclosure is a stent formed in a tubular shape by braiding one or more wire rods.
A radial force (RF) ranges from 0.02 N/mm to 0.04 N/mm, and
a ratio (RF/AF) of the radial force (RF) to an axial force (AF) is 0.14 mm⁻¹ or more.
(2) The stent of the disclosure preferably has a shortening of 35% or less.
Known stents do not fulfill the above conditions, and only the stent of the disclosure fulfills the conditions.
(3) The axial force (AF) of the stent of the disclosure is preferably 0.3 N or less.
(4) The stent of the disclosure is preferably made of a structure formed by the wire rod, including, along an axial direction, a plurality of circumferential units in which a plurality of meshes is arrayed along a circumferential direction, and including a bent part of one of adjacent circumferential units is coupled to a bent part or a wire rod intersecting part of the other of adjacent circumferential units.
(5) In the stent of (4) above, preferably, the wire rod forming the structure has a diameter of from 0.1 mm to 0.5 mm, in the structure, the number of coupling points per unit area of the bent part of the one of adjacent circumferential units and the bent part or the wire rod intersecting part of the other of adjacent circumferential units ranges from 2 points/cm² to 8 points/cm², and
the number of the coupling points per unit length in the axial direction arrayed at the same circumferential position is 2 points/cm or more.

Forming the structure with the wire rod having a diameter of from 0.1 mm to 0.5 mm and setting the number of the coupling points per unit area in the structure to from 2 points/cm² to 8 points/cm² allows the radial force (RF) to range from 0.02 N/mm to 0.04 N/mm.

In addition to setting the number of the coupling points per unit area within the above-described range, setting the number of the coupling points per unit length in the axial direction arrayed at the same circumferential position to 2 points/cm or more allows the axial force (AF) to be 0.3 N or less and the ratio (RF/AF) to be 0.14 mm⁻¹ or more.
(6) The stent of the disclosure is preferably made of: a first mesh structure formed of a first wire rod and including, along an axial direction, a plurality of circumferential units in which a plurality of meshes is arrayed along a circumferential direction; and
a second mesh structure formed of a second wire rod, including, along the axial direction, a plurality of circumferential units in which a plurality of meshes is arrayed along the circumferential direction, and braided with the first mesh structure.

In the first mesh structure, a bent part of one of adjacent circumferential units is coupled to a bent part or a wire rod intersecting part of the other of adjacent circumferential units, and in the second mesh structure, a bent part of one of adjacent circumferential units is coupled to none of a bent part of the other of adjacent circumferential units and a wire rod intersecting part.
(7) In the stent according to (6), preferably, the first wire rod has a diameter of from 0.1 mm to 0.5 mm, in the first mesh structure, the number of coupling points per unit area of the bent part of the one of adjacent circumferential units and the bent part or the wire rod intersecting part of the other of adjacent circumferential units ranges from 2 points/cm² to 8 points/cm², and
the number of the coupling points per unit length in the axial direction arrayed at the same circumferential position is 2 points/cm or more.

Forming the first mesh structure with the first wire rod having a diameter of from 0.1 mm to 0.5 mm and setting the number of the coupling points per unit area in the first mesh structure to from 2 points/cm² to 8 points/cm² allows the radial force (RF) of the stent including the second mesh structure to range from 0.02 N/mm to 0.04 N/mm.

In the first mesh structure, in addition to setting the number of the coupling points per unit area within the above-described range, setting the number of the coupling points per unit length in the axial direction arrayed at the same circumferential position to 2 points/cm or more allows the axial force (AF) of the stent to be 0.3 N or less and the ratio (RF/AF) to be 0.14 mm⁻¹ or more.

The stent of the disclosure has, in a well-balanced manner, a high expansion force that enables the stent to come into close contact with an inner wall of a tubular organ to sufficiently expand a constricted part and good flexibility by which none of an ulcer and a perforation are generated even when the stent is placed in a bent tubular organ.

### BRIEF DESCRIPTION OF DRAWINGS

FIG. 1 is a development view illustrating a main part of a stent according to a first embodiment of the disclosure.

FIG. 2 is a development view schematically illustrating a first mesh structure of the stent according to the first embodiment.

FIG. 3 is a development view illustrating a main part of a stent according to a second embodiment of the disclosure.

FIG. 4 is a development view schematically illustrating a first mesh structure of the stent according to the second embodiment.

FIG. 5 is a schematic view for explaining a measurement method of an axial force (AF) defined in the disclosure.

### DESCRIPTION OF EMBODIMENTS

### First Embodiment

Hereinafter, specific embodiments of the disclosure will be described in detail.

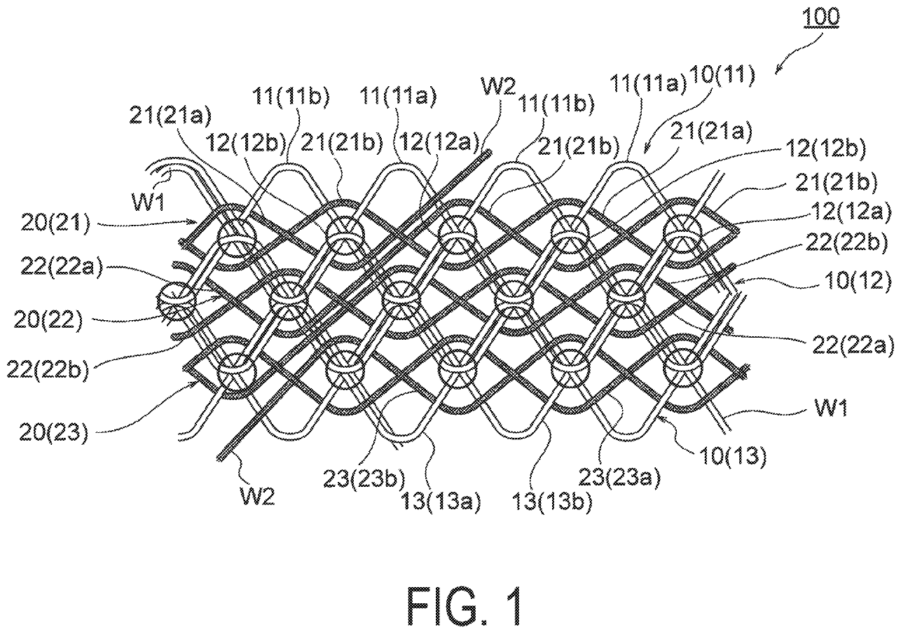

A stent 100 of the present embodiment illustrated in FIG. 1 is made of a first mesh structure 10 provided along an axial direction with adjacent circumferential units of a circumferential unit 11 of a first stage, a circumferential unit 12 of a second stage, and a circumferential unit 13 of a third stage having an overlapping part and a second mesh structure 20 provided along an axial direction with adjacent circumferential units of a circumferential unit 21 of a first stage, a circumferential unit 22 of a second stage, and a circumferential unit 23 of a third stage that do not overlap each other. A radial force (RF) of the stent 100 ranges from 0.02 N/mm to 0.04 N/mm, an axial force (AF) is 0.3 N or less, a ratio (RF/AF) of the radial force to the axial force is 0.14 mm⁻¹ or more, and shortening is 35% or less.

The circumferential unit 11 of the first stage constituting the first mesh structure 10 is formed of a first loop 11a formed by advancing a first wire rod W1 along the circumferential direction while folding back the first wire rod W1 right and left, and a second loop 11b that is continuous with the first loop 11a and is formed by advancing the first wire rod W1 along the circumferential direction while folding back the first wire rod W1 right and left with a phase shifted by ½ pitches with respect to the first loop 11a.

The second loop 11b intersects a straight part of the first loop 11a in a cross shape and advances alternately passing above and below the first loop 11a.

The circumferential unit 12 of the second stage constituting the first mesh structure 10 is shifted by ½ pitches (½ of the amplitude corresponding to the axial length of the circumferential unit 11) in the axial direction with respect to the circumferential unit 11 of the first stage adjacent to the circumferential unit 12 and shifted by ¼ pitches in the circumferential direction.

The circumferential unit 12 is formed of a first loop 12a that is continuous with the second loop 11b of the circumferential unit 11 of the first stage and is formed by advancing the first wire rod W1 along the circumferential direction while folding back the first wire rod W1 right and left and a second loop 12b that is continuous with the first loop 12a and is formed by advancing the first wire rod W1 along the circumferential direction while folding back the first wire rod W1 right and left so that the phase is shifted by ½ pitches with respect to the first loop 12a.

The second loop 12b intersects a straight part of the first loop 12a in a cross shape and advances alternately passing above and below the first loop 12a.

The circumferential unit 12 of the second stage shifted by ½ pitches in the axial direction with respect to the circumferential unit 11 of the first stage has a part overlapping the circumferential unit 11.

The bent part of the circumferential unit 12 shifted by ¼ pitches in the circumferential direction with respect to the circumferential unit 11 of the first stage is coupled to the wire rod intersecting part of the circumferential unit 11, and the wire rod intersecting part of the circumferential unit 12 is coupled to the bent part of the circumferential unit 11.

The circumferential unit 13 of the third stage constituting the first mesh structure 10 is shifted by ½ pitches in the axial direction with respect to the circumferential unit 12 of the second stage adjacent to the circumferential unit 13 and shifted by ¼ pitches in the circumferential direction.

The circumferential unit 13 is formed of a first loop 13a that is continuous with the second loop 12b of the circumferential unit 12 of the second stage and is formed by advancing the first wire rod W1 along the circumferential direction while folding back the first wire rod W1 right and left and a second loop 13b that is continuous with the first loop 13a and is formed by advancing the first wire rod W1 along the circumferential direction while folding back the first wire rod W1 right and left with a phase shifted by ½ pitches with respect to the first loop 13a.

The second loop 13b intersects a straight part of the first loop 13a in a cross shape and advances alternately passing above and below the first loop 13a.

The circumferential unit 13 of the third stage shifted by ½ pitches in the axial direction with respect to the circumferential unit 12 of the second stage has a part overlapping the circumferential unit 12.

The bent part of the circumferential unit 13 shifted by ¼ pitches in the circumferential direction with respect to the circumferential unit 12 of the second stage is coupled to the wire rod intersecting part of the circumferential unit 12, and the wire rod intersecting part of the circumferential unit 13 is coupled to the bent part of the circumferential unit 12.

FIG. 1 illustrates only the circumferential units of the three stages (the circumferential unit 11, the circumferential unit 12, and the circumferential unit 13) as the circumferential units constituting the first mesh structure 10, but the first mesh structure 10 of the stent 100 of the present embodiment is usually provided with circumferential units of the fourth and subsequent stages along the axial direction.

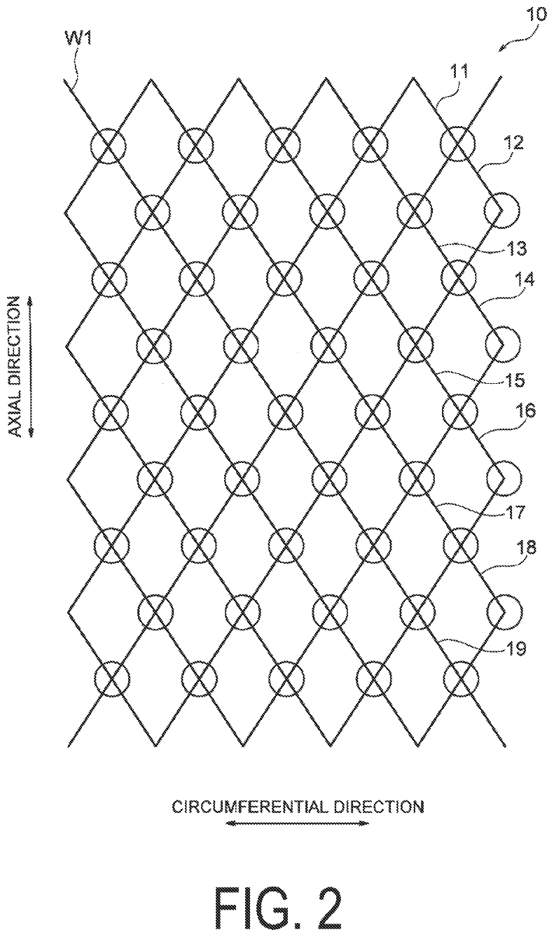

FIG. 2 illustrates the first mesh structure 10 provided with circumferential units of nine stages of (circumferential units 11 to 19). In the figure, a part where adjacent circumferential units (wire rods) overlap each other is also indicated by one line.

The circumferential unit of the nth stage (n≥4) constituting the first mesh structure 10 is shifted by ½ pitches in the axial direction with respect to the circumferential unit of the (n−1)th stage adjacent to the circumferential unit of the nth stage and shifted by ¼ pitches in the circumferential direction.

The circumferential unit of the nth stage is formed of a first loop that is continuous with a second loop of the circumferential unit of the (n−1)th stage and is formed by advancing the first wire rod W1 along the circumferential direction while folding back the first wire rod W1 right and left and a second loop that is continuous with the first loop and is formed by advancing the first wire rod W1 along the circumferential direction while folding back the first wire rod W1 right and left with a phase shifted by ½ pitches with respect to the first loop.

The second loop intersects a straight part of the first loop in a cross shape, and advances alternately passing above and below the first loop.

The circumferential unit of the nth stage shifted by ½ pitches in the axial direction with respect to the circumferential unit of the (n−1)th stage has a part overlapping the circumferential unit of the (n−1)th stage.

The bent part of the circumferential unit of the nth stage shifted by ¼ pitches in the circumferential direction with respect to the circumferential unit of the (n−1)th stage is coupled to the wire rod intersecting part of the circumferential unit of the (n−1)th stage, and the wire rod intersecting part of the circumferential unit of the nth stage is coupled to the bent part of the circumferential unit of the (n−1)th stage.

The diameter of the first wire rod W1 forming the first mesh structure 10 ranges from 0.1 mm to 0.5 mm and preferably from 0.15 mm to 0.25 mm.

When the diameter of the first wire rod W1 is excessively small, it is difficult to set the radial force (RF) of the resulting stent to 0.02 N/mm or more.

On the other hand, when the diameter of the first wire rod W1 is excessively large, it is difficult to set the radial force (RF) of the resulting stent to 0.04 N/mm or less.

In the first mesh structure 10, a bent part of one of adjacent circumferential units is coupled to a wire rod intersecting part of the other of adjacent circumferential units, and FIGS. 1 and 2 illustrate these coupling points surrounded by “o”.

In the first mesh structure 10, the number of the coupling points per unit area preferably ranges from 2 points/cm² to 8 points/cm² and more preferably from 3/cm² to 5/cm².

As illustrated in FIG. 1, the wire rod intersecting part of the circumferential unit 12 is coupled to the bent part of the circumferential unit 11 and is also coupled to the bent part of the circumferential unit 13, and the number of coupling points in this case is counted as one.

Setting the number of coupling points per unit area to 2 points/cm² or more allows the radial force (RF) of the stent 100 to be 0.02 N/mm or more.

Setting the number of coupling points per unit area to 8 points/cm² or less allows the radial force (RF) of the stent 100 to be 0.04 N/mm or less.

In the first mesh structure 10, the number of the coupling points per unit length in the axial direction arrayed at the same circumferential position is preferably 2/cm or more and more preferably ranges from 2 points/cm to 3 points/cm.

Setting the number of coupling points per unit length in the axial direction to 2 points/cm or more allows the stent 100 to be easily bent and the axial force (AF) thereof to be 0.3 N or less and the ratio (RF/AF) to be 0.14 mm⁻¹ or more.

The number of coupling points per unit length in the axial direction is preferably 5 points/cm or less from the viewpoint of reducing the elongation allowance of the first mesh structure 10 in the axial direction and setting the shortening of the stent 100 to 35% or less.

The circumferential unit 21 of the first stage constituting the second mesh structure 20 is formed of a first loop 21a formed by advancing a second wire rod W2 along the circumferential direction while folding back the second wire rod W2 right and left while having a pitch length same as the circumferential unit 11 of the first mesh structure 10 corresponding to the circumferential unit 21, having an amplitude smaller than the corresponding circumferential unit 11, and being shifted by ¼ pitches in the circumferential direction with respect to the corresponding circumferential unit 11 and a second loop 21b that is continuous with the first loop 21a and is formed by advancing the second wire rod W2 along the circumferential direction while folding back the second wire rod W2 right and left with a phase shifted by ½ pitches with respect to the first loop 21a.

The second loop 21b intersects a straight part of the first loop 21a in a cross shape and advances alternately passing above and below the first loop 21a.

The circumferential unit 21 is braided with the circumferential unit 11 of the corresponding first mesh structure 10. The bent part of the circumferential unit 21 is coupled (engaged) with the bent part of the circumferential unit 12 of the first mesh structure 10.

The circumferential unit 22 of the second stage constituting the second mesh structure 20 is formed of a first loop 22a formed by advancing the second wire rod W2 along the circumferential direction while folding back the second wire rod W2 right and left while having a pitch length same as the circumferential unit 12 of the first mesh structure 10 corresponding to the circumferential unit 22, having an amplitude smaller than the corresponding circumferential unit 12, and being shifted by ¼ pitches in the circumferential direction with respect to the corresponding circumferential unit 12 and a second loop 22b that is continuous with the first loop 22a and is formed by advancing the second wire rod W2 along the circumferential direction while folding back the second wire rod W2 right and left so that the phase is shifted by ½ pitches with respect to the first loop 22a.

The second loop 22b intersects a straight part of the first loop 22a in a cross shape and advances alternately passing above and below the first loop 22a.

The circumferential unit 22 is braided with the circumferential unit 12 of the corresponding first mesh structure 10. The bent part of the circumferential unit 22 is coupled (engaged) with the bent part of the circumferential unit 11 of the first mesh structure 10, and is also coupled (engaged) with the bent part of the circumferential unit 13 of the first mesh structure 10.

The circumferential unit 22 and the circumferential unit 21 have no overlapping part, and the bent part of the circumferential unit 22 is coupled to none of the bent part and the wire rod intersecting part of the circumferential unit 21 of the first stage.

The circumferential unit 23 of the third stage constituting the second mesh structure 20 is formed of a first loop 23a formed by advancing the second wire rod W2 along the circumferential direction while folding back the second wire rod W2 right and left while having a pitch length same as the circumferential unit 13 of the first mesh structure 10 corresponding to the circumferential unit 23, having an amplitude smaller than the corresponding circumferential unit 13, and being shifted by ¼ pitches in the circumferential direction with respect to the corresponding circumferential unit 13 and a second loop 23b that is continuous with the first loop 23a and is formed by advancing the second wire rod W2 along the circumferential direction while folding back the second wire rod W2 right and left so that the phase is shifted by ½ pitches with respect to the first loop 23a.

The second loop 23b intersects a straight part of the first loop 23a in a cross shape and advances alternately passing above and below the first loop 23a.

The circumferential unit 23 is braided with the circumferential unit 13 of the corresponding first mesh structure 10. The bent part of the circumferential unit 23 is coupled (engaged) with the bent part of the circumferential unit 12 of the first mesh structure 10.

The circumferential unit 23 and the circumferential unit 22 have no overlapping part, and the bent part of the circumferential unit 23 is coupled to none of the bent part and the wire rod intersecting part of the circumferential unit 22 of the second stage.

FIG. 1 illustrates only the circumferential units of the three stages (the circumferential unit 21, the circumferential unit 22, and the circumferential unit 23) as the circumferential units constituting the second mesh structure 20, but the second mesh structure 20 of the stent 100 of the present embodiment is usually provided with circumferential units of the fourth and subsequent stages along the axial direction.

The circumferential unit of the nth stage (n≥4) constituting the second mesh structure 20 is formed of a first loop formed by advancing the second wire rod W2 along the circumferential direction while folding back the second wire rod W2 right and left while having a pitch length same as the circumferential unit of the nth stage constituting the first mesh structure 10 corresponding to the circumferential unit of the nth stage, having an amplitude smaller than the circumferential unit of the nth stage constituting the first mesh structure 10, and being shifted by ¼ pitches in the circumferential direction with respect to the circumferential unit of the nth stage constituting the first mesh structure 10 and a second loop that is continuous with the first loop and is formed by advancing the second wire rod W2 along the circumferential direction while folding back the second wire rod W2 right and left so that a phase is shifted by ½ pitches with respect to the first loop.

The second loop intersects a straight part of the first loop in a cross shape and advances alternately passing above and below the first loop.

The circumferential unit of the nth stage constituting the second mesh structure 20 is braided with the circumferential unit of the nth stage constituting the first mesh structure 10. The bent part of the circumferential unit of the nth stage constituting the second mesh structure 20 is coupled (engaged) with the bent part of the circumferential unit of the (n−1)th step constituting the first mesh structure 10.

The circumferential unit of the nth stage and the circumferential unit of the (n−1)th stage constituting the second mesh structure 20 have no overlapping part, and the bent part of the circumferential unit of the nth stage is coupled to none of the bent part and the wire rod intersecting part in the circumferential unit of the (n−1)th stage.

The diameter of the second wire rod W2 forming the second mesh structure 20 ranges from 0.1 mm to 0.5 mm and preferably from 0.15 mm to 0.25 mm.

In the second mesh structure 20, since a bent part of one of adjacent circumferential units is coupled to none of a bent part and the wire rod intersecting part of the other of adjacent circumferential units (no coupling point as in the first mesh structure 10 exists), the form of the second mesh structure 20 does not substantially affect the radial force (RF), the axial force (AF), and the shortening of the stent 100.

The outer diameter of the stent 100 of the present embodiment ranges from, for example, 10 mm to 40 mm, preferably from 15 mm to 30 mm, and more preferably from 16 mm to 25 mm.

The outer diameter of the stent 100 may be the same over the entire length, or one end part and/or the other end part may be enlarged.

The length of the stent 100 ranges from, for example, 40 mm to 200 mm, preferably from 50 mm to 180 mm, and more preferably from 60 mm to 150 mm.

The radial force (RF) of the stent 100 of the present embodiment ranges from 0.02 N/mm to 0.04 N/mm and preferably from 0.025 N/mm to 0.037 N/mm.

The radial force (RF) in the range of 0.02 N/mm to 0.04 N/mm allows the stent 100 to come into close contact with a constricted part to sufficiently expand the constricted part without damaging the inner wall of the digestive tract constricted by a tumor.

If the radial force (RF) is less than 0.02 N/mm, it is not possible to bring the stent 100 into close contact with the inner wall of the digestive tract and sufficiently expand the constricted part.

On the other hand, if the radial force (RF) exceeds 0.04 N/mm, there is a risk of damaging the inner wall of the digestive tract.

The radial force (RF) defined in the disclosure is measured as follows in compliance with JIS T 0401 (Mechanical testing methods for stent grafts), 4.2 Measurement device for force in radial direction (FIG. 2), and JIS T 3269 (Stents and drainage catheters for biliary and pancreatic ducts) Annex A (Confirmation test for dynamic characteristics) A.4 Procedure a) Test method 1.

With the temperature set to 37±2° C., a compressive load is applied perpendicularly to the axial direction of the stent, the compression is performed up to 50% of an initial diameter, then the load is removed at a constant speed to restore the stent diameter, and a value F/L [N/mm] obtained by dividing a load F when the stent diameter becomes 70% of the initial diameter by an axial length L in the range applied with the load is defined as the radial force (RF).

The axial force (AF) of the stent 100 of the present embodiment is 0.3 N or less, preferably 0.21 or less, and the ratio (RF/AF) of the radial force to the axial force is 0.14 mm⁻¹ or more and preferably 0.15 mm⁻¹ or more.

Since the axial force (AF) is 0.3 N or less and the ratio (RF/AF) is 0.14 mm⁻¹ or more, the stent 100 has, in a well-balanced manner, a high expansion force that enables the stent 100 to sufficiently expand the constricted part of the digestive tract and good flexibility by which none of an ulcer and a perforation is generated at sites with which both end parts of the stent come into contact even when the stent is placed in the bent digestive tract.

If the ratio (RF/AF) is less than 0.14 mm, both end parts of the stent press the inner wall of the digestive tract to cause an ulcer or a perforation (when AF is excessively large), or it is not possible to bring the stent 100 into close contact with the inner wall of the digestive tract and sufficiently expand the constricted part (when RF is excessively small).

The axial force (AF) defined in the disclosure is measured as follows in compliance with Gastrointest Endosc. 2009 July; 70(1):37-44. Measurement of radial and axial forces of biliary self-expandable metallic stents Hiroyuki Isayama et. al.

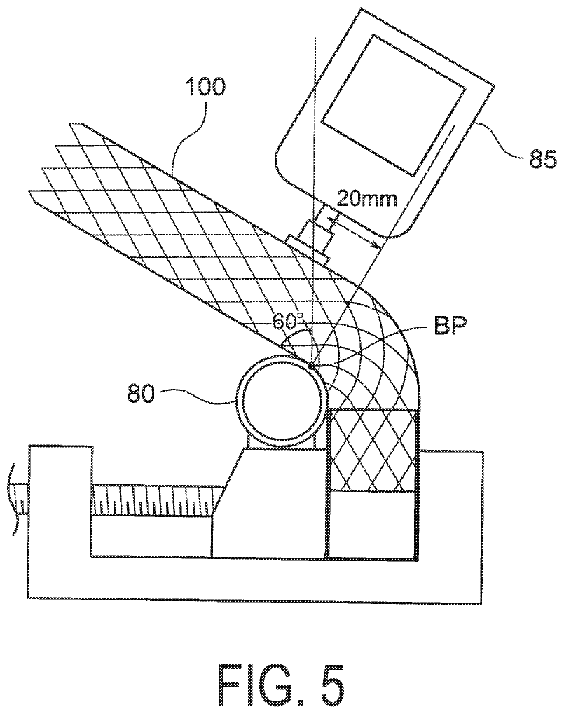

With the temperature set to 37±2° C., by applying a load perpendicularly to the axial direction to the stent in an upright state with a lower end part fixed, as illustrated in FIG. 5, the stent is bent by 60° along a pipe 80 having the same outer diameter as the stent 100, and a reaction force [N] measured by a digital force gauge 85 at a position 20 mm away from a bending point (BP) is defined as the axial force (AF).

The shortening of the stent 100 of the present embodiment is 35% or less, and preferably 30% or less.

Since the shortening is 35% or less, the stent 100 can be accurately placed at the target site.

According to the stent 100 of the present embodiment, it is possible to sufficiently expand the constricted part of the digestive tract, and even when the stent is placed in the bent digestive tract, an ulcer or a perforation does not occur at sites with which both end parts of the stent come into contact.

Since each of the circumferential units of the first mesh structure 10 and each of the circumferential units of the second mesh structure 20 have the same pitch length and the phases being shifted by ¼ pitches, the area of the mesh in each of the circumferential units of the first mesh structure 10 can be divided into four by the second wire rod W2 constituting each of the circumferential units of the second mesh structure 20, and therefore the mesh can be made finer than that of a stent including only the first mesh structure 10.

Since each of the circumferential units of the second mesh structure 20 is shifted by ¼ pitches in the circumferential direction with respect to each of the circumferential units of the first mesh structure 10, the bent part of the circumferential unit of the first mesh structure 10 and the bent part of the circumferential unit of the second mesh structure 20 are not at the same circumferential position, and a coupling part by the bent part of the circumferential unit of the first mesh structure 10 and a coupling part by the bent part of the circumferential unit of the second mesh structure 20 are not arrayed at the same circumferential position, it is possible to avoid the followability to a curved shape of the tubular organ from being impaired.

Since the amplitude of each of the circumferential units of the second mesh structure 20 is smaller than the amplitude of each of the circumferential units of the first mesh structure 10, the bent part of the circumferential unit of the first mesh structure 10 and the bent part of the circumferential unit of the second mesh structure 20 are not at the same axial position, and the coupling part by the bent part of the circumferential unit of the first mesh structure 10 and the coupling part by the bent part of the circumferential unit of the second mesh structure 20 are not arrayed at the same axial position, and therefore it is possible to avoid the diameter reduction property of the stent from being impaired.

### Second Embodiment

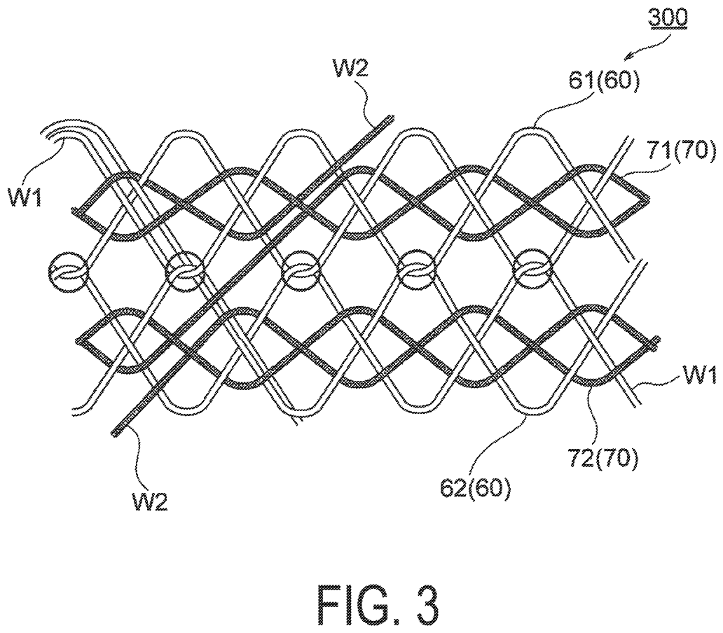

A stent 300 of the present embodiment illustrated in FIG. 3 is made of a first mesh structure 60 provided with a circumferential unit 61 of the first stage and a circumferential unit 62 of the second stage along the axial direction, and a second mesh structure 70 provided with a circumferential unit 71 of the first stage and a circumferential unit 72 of the second stage along the axial direction, in which a radial force (RF) of the stent 100 ranges from 0.02 N/mm to 0.04 N/mm, an axial force (AF) is 0.3 N or less, a ratio (RF/AF) of the radial force to the axial force is 0.14 mm⁻¹ or more, and shortening is 35% or less.

The circumferential units (circumferential units 61 and 62) of the first mesh structure 60 and the circumferential units (circumferential units 71 and 72) of the second mesh structure 70 each includes two loops, the two loops intersect a straight part of each other in a cross shape, and one loop advances alternately passing above and below the other loop.

As illustrated in FIG. 3, the circumferential unit 61 of the first stage and the circumferential unit 62 of the second stage constituting the first mesh structure 60 are shifted by one pitch (amplitude) in the axial direction, and the bent part of the circumferential unit 61 is coupled (engaged) with the bent part of the circumferential unit 62.

FIG. 3 illustrates only the circumferential units of the two stages (the circumferential unit 61 and the circumferential unit 62) as the circumferential units constituting the first mesh structure 60, but the first mesh structure 60 of the stent 300 of the present embodiment is usually provided with circumferential units of the third and subsequent stages along the axial direction.

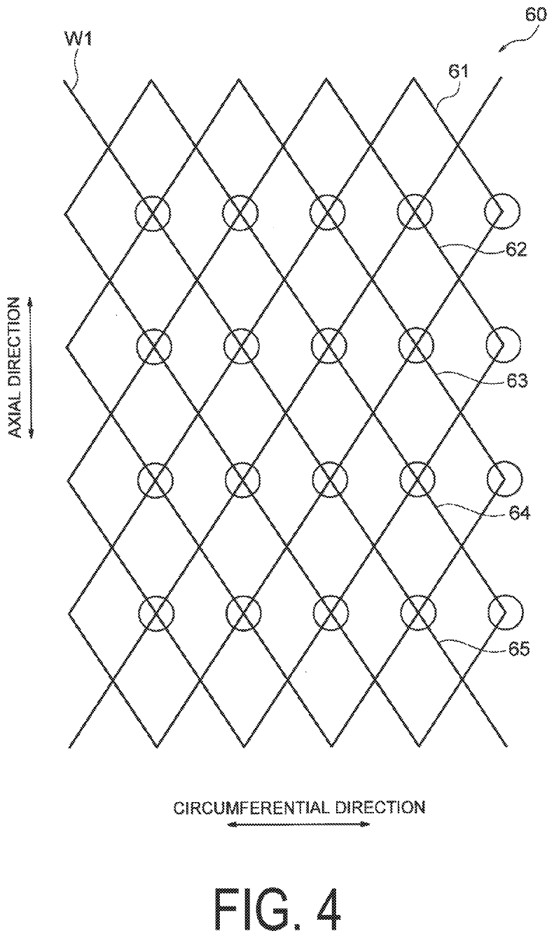

FIG. 4 illustrates the first mesh structure 60 including circumferential units (circumferential units 61 to 65) of five stages.

The circumferential unit of the (n−1)th stage and the circumferential unit of the nth stage constituting the first mesh structure 60 are shifted by one pitch (amplitude) in the axial direction, and the bent part of the circumferential unit of the (n−1)th stage is coupled (engaged) with the bent part of the circumferential unit of the nth stage.

The diameter of the first wire rod W1 forming the first mesh structure 60 ranges from 0.1 mm to 0.5 mm and preferably 0.15 mm to 0.25 mm.

When the diameter of the first wire rod W1 is excessively small, it is difficult to set the radial force (RF) of the resulting stent to 0.02 N/mm or more.

On the other hand, when the diameter of the first wire rod W1 is excessively large, it is difficult to set the radial force (RF) of the resulting stent to 0.04 N/mm or less.

In the first mesh structure 60, a bent part of one of adjacent circumferential units is coupled to a wire rod intersecting part of the other of adjacent circumferential units, and FIGS. 3 and 4 illustrate these coupling points surrounded by “◯”.

In the first mesh structure 60, the number of the coupling points per unit area ranges from preferably 2 points/cm² to 8 points/cm² and more preferably from 3 points/cm² to 5 points/cm².

Setting the number of coupling points per unit area to 2 points/cm² or more allows the radial force (RF) of the stent 100 to be 0.02 N/mm or more.

Setting the number of coupling points per unit area to 8 points/cm² or less allows the radial force (RF) of the stent 100 to be 0.04 N/mm or less.

In the first mesh structure 60, the number of the coupling points per unit length in the axial direction arrayed at the same circumferential position preferably ranges from 2 points/cm or more and more preferably from 2 points/cm to 3 points/cm.

Setting the number of coupling points per unit length in the axial direction to 2 points/cm or more allows the stent 100 to be easily bent and the axial force (AF) thereof to be 0.3 N or less and the ratio (RF/AF) to be 0.14 mm⁻¹ or more.

The number of coupling points per unit length in the axial direction is preferably 5 points/cm or less from the viewpoint of reducing the elongation allowance of the first mesh structure 60 in the axial direction and setting the shortening of the stent 100 to 35% or less.

The circumferential unit 71 of the first stage constituting the second mesh structure 70 is formed with a pitch length same as the circumferential unit 61 of the first mesh structure 60 corresponding to the circumferential unit 71 and an amplitude smaller than the circumferential unit 61 and is braided with the circumferential unit 61 while being shifted by ¼ pitches in the circumferential direction with respect to the circumferential unit 61.

The circumferential unit 71 has no part overlapping the circumferential unit 62 of the first mesh structure 60, and the bent part of the circumferential unit 71 is coupled to none of the bent part and the wire rod intersecting part of the circumferential unit 62.

The circumferential unit 72 of the second stage constituting the second mesh structure 70 is continuous with the end part of the circumferential unit 71, is formed with a pitch length same as the circumferential unit 62 of the first mesh structure 60 corresponding to the circumferential unit 72 and an amplitude smaller than the circumferential unit 62, and is braided with the circumferential unit 62 while being shifted by ¼ pitches in the circumferential direction with respect to the circumferential unit 62.

The circumferential unit 72 has no part overlapping the circumferential unit 61 of the first mesh structure 60, and the bent part of the circumferential unit 72 is coupled to none of the bent part and the wire rod intersecting part of the circumferential unit 61.

The circumferential unit 72 has no part overlapping the circumferential unit 71 of the second mesh structure 70, and the bent part of the circumferential unit 72 is coupled to none of the bent part and the wire rod intersecting part of the circumferential unit 71.

FIG. 3 illustrates only the circumferential units of the two stages (the circumferential unit 71 and the circumferential unit 72) as the circumferential units constituting the second mesh structure 70, but the second mesh structure 70 of the stent 300 of the present embodiment is usually provided with circumferential units of the third and subsequent stages along the axial direction.

The circumferential unit of the nth stage constituting the second mesh structure 70 is continuous with the end part of the circumferential unit of the (n−1)th stage constituting the second mesh structure 70, is formed with a pitch length same as the circumferential unit of the nth stage constituting the first mesh structure 60 and an amplitude smaller than the circumferential unit of the nth stage constituting the first mesh structure 60, and is braided with the circumferential unit of the nth stage constituting the first mesh structure 60 while being shifted by ¼ pitches in the circumferential direction with respect to the circumferential unit of the nth stage constituting the first mesh structure 60.

The circumferential unit of the nth stage constituting the second mesh structure 70 has no part overlapping the circumferential unit of the (n−1)th stage constituting the first mesh structure 60, and the bent part of the circumferential unit of the nth stage constituting the second mesh structure 70 is coupled to none of the bent part and the wire rod intersecting part of the circumferential unit of the (n−1)th stage constituting the first mesh structure 60.

The circumferential unit of the nth stage constituting the second mesh structure 70 has no part overlapping the circumferential unit of the (n−1)th stage constituting the second mesh structure 70, and the bent part of the circumferential unit of the nth stage constituting the second mesh structure 70 is coupled to none of the bent part and the wire rod intersecting part of the circumferential unit of the (n−1)th stage constituting the second mesh structure 70.

The diameter of the second wire rod W2 forming the second mesh structure 70 ranges from 0.1 mm to 0.5 mm and preferably from 0.15 mm to 0.25 mm.

In the second mesh structure 70, since a bent part of one of adjacent circumferential units is coupled to none of a bent part and the wire rod intersecting part of the other of adjacent circumferential units (no coupling point as in the first mesh structure 60 exists), the form of the second mesh structure 70 does not substantially affect the radial force (RF), the axial force (AF), and the shortening of the stent 300.

The outer diameter of the stent 300 of the present embodiment ranges from, for example, 10 mm to 40 mm, preferably from 15 mm to 30 mm, and more preferably from 16 mm to 25 mm.

The outer diameter of the stent 300 may be the same over the entire length, or one end part and/or the other end part may be enlarged.

The length of the stent 300 ranges from, for example, 40 mm to 200 mm, preferably from 50 mm to 180 mm, and more preferably from 60 mm to 150 mm.

The radial force (RF) of the stent 300 of the present embodiment ranges from 0.02 N/mm to 0.04 N/mm and preferably from 0.025 N/mm to 0.037 N/mm.

The radial force (RF) in the range of 0.02 N/mm to 0.04 N/mm allows the stent 300 to come into close contact with a constricted part and sufficiently expand the constricted part without damaging the inner wall of the digestive tract constricted by a tumor.

The axial force (AF) of the stent 300 of the present embodiment is 0.3 N or less, preferably 0.21 or less, and the ratio (RF/AF) of the radial force to the axial force is 0.14 mm⁻¹ or more and preferably 0.15 mm⁻¹ or more.

Since the axial force (AF) is 0.3 N or less and the ratio (RF/AF) is 0.14 mm⁻¹ or more, the stent 300 has, in a well-balanced manner, a high expansion force that enables the stent 300 to sufficiently expand the constricted part of the digestive tract and good flexibility by which none of an ulcer and a perforation is generated at sites with which both end parts of the stent come into contact even when the stent is placed in the bent digestive tract.

The shortening of the stent 300 of the present embodiment is 35% or less, and preferably 30% or less.

Since the shortening is 35% or less, the stent 300 can be accurately placed at the target site.

According to the stent 300 of the present embodiment, it is possible to sufficiently expand the constricted part of the digestive tract, and even when the stent is placed in the bent digestive tract, an ulcer or a perforation does not occur at sites with which both end parts of the stent come into contact.

Since each of the circumferential units of the first mesh structure 60 and each of the circumferential units of the second mesh structure 70 have the same pitch length and the phases being shifted by ¼ pitches, the area of the mesh in each of the circumferential units of the first mesh structure 60 can be divided into four by the second wire rod W2 constituting each of the circumferential units of the second mesh structure 70, and therefore the mesh can be made finer than that of a stent including only the first mesh structure 60.

Since each of the circumferential units of the second mesh structure 70 is shifted by ¼ pitches in the circumferential direction with respect to each of the circumferential units of the first mesh structure 60, the bent part of the circumferential unit of the first mesh structure 60 and the bent part of the circumferential unit of the second mesh structure 70 are not at the same circumferential position, and a coupling part by the bent part of the circumferential unit of the first mesh structure 60 and a coupling part by the bent part of the circumferential unit of the second mesh structure 70 are not arrayed at the same circumferential position, it is possible to avoid the followability to a curved shape of the tubular organ from being impaired.

Since the amplitude of each of the circumferential units of the second mesh structure 70 is smaller than the amplitude of each of the circumferential units of the first mesh structure 60, the bent part of the circumferential unit of the first mesh structure 60 and the bent part of the circumferential unit of the second mesh structure 70 are not at the same axial position, and the coupling part by the bent part of the circumferential unit of the first mesh structure 60 and the coupling part by the bent part of the circumferential unit of the second mesh structure 70 are not arrayed at the same axial position, and therefore it is possible to avoid the diameter reduction property of the stent from being impaired.

While preferred embodiments of the disclosure have been described above, it is to be understood that variations and modifications will be apparent to those skilled in the art without departing from the scope and spirit of the disclosure. The scope of the disclosure, therefore, is to be determined solely by the following claims.

1. A stent formed in a tubular shape by braiding one or more wire rods, wherein
a radial force (RF) ranges from 0.02 N/mm to 0.04 N/mm, and
a ratio (RF/AF) of the radial force (RF) to an axial force (AF) is 0.14 mm⁻¹ or more.

2. The stent according to claim 1, wherein shortening is 35% or less.

3. The stent according to claim 1, wherein the axial force (AF) is 0.3 N or less.

4. The stent according to claim 1 made of a structure formed of the wire rod, the structure including, along an axial direction, a plurality of circumferential units in which a plurality of meshes is arrayed along a circumferential direction and including a bent part of one of adjacent circumferential units is coupled to a bent part or a wire rod intersecting part of the other of adjacent circumferential units.

5. The stent according to claim 4, wherein
the wire rod forming the structure has a diameter of from 0.1 mm to 0.5 mm,
in the structure, the number of coupling points per unit area of the bent part of the one of adjacent circumferential units and the bent part or the wire rod intersecting part of the other of adjacent circumferential units ranges from 2 points/cm² to 8 points/cm², and
the number of the coupling points per unit length in the axial direction arrayed at the same circumferential position is 2 points/cm or more.

6. The stent according to claim 1 made of:
a first mesh structure formed of a first wire rod, the first mesh structure including, along an axial direction, a plurality of circumferential units in which a plurality of meshes is arrayed along a circumferential direction; and
a second mesh structure formed of a second wire rod, the second mesh structure including, along the axial direction, a plurality of circumferential units in which a plurality of meshes is arrayed along the circumferential direction and braided with the first mesh structure, wherein
in the first mesh structure, a bent part of one of adjacent circumferential units is coupled to a bent part or a wire rod intersecting part of the other of adjacent circumferential units, and
in the second mesh structure, a bent part of one of adjacent circumferential units is coupled to none of a bent part and a wire rod intersecting part of the other of adjacent circumferential units.

7. The stent according to claim 6, wherein
the first wire rod has a diameter of from 0.1 mm to 0.5 mm,
in the first mesh structure, the number of coupling points per unit area of the bent part of the one of adjacent circumferential units and the bent part or the wire rod intersecting part of the other of adjacent circumferential units ranges from 2 points/cm² to 8 points/cm², and
the number of the coupling points per unit length in the axial direction arrayed at the same circumferential position is 2 points/cm or more.
